(19) United States
(12) Patent Application Publication
(10) Pub. No.: US 2006/0008497 A1
(43) Pub. Date: Jan. 12, 2006

(54) Implantable apparatus having improved biocompatibility and process of making the same

(76) Inventor: Shlomo Gabbay, Short Hills, NJ (US)

Correspondence Address: TAROLLI, SUNDHEIM, COVELL & TUMMINO L.L.P., 526 SUPERIOR AVENUE, SUITE 1111, CLEVEVLAND, OH, 44114, US

(21) Appl. No.: 10/888,876

(22) Filed: Jul. 9, 2004

(51) Int. Cl. A61F 13/00

(52) U.S. Cl. 424/422

(57) ABSTRACT

An implantable apparatus has a plurality of fixed and detoxified protein layers formed in situ overlying at least one surface to improve biocompatibility. A process of making the implantable apparatus can include repeatedly exposing at least one surface of the implantable apparatus to a protein solution to form a multi-layered coating of protein on the at least one surface and fixing the protein layered implantable apparatus with a fixation solution and then substantially detoxifying the multi-layered protein coating.

### TECHNICAL FIELD

The present invention relates to an implantable apparatus and more particularly to an implantable apparatus having improved biocompatibility and to a process of making an implantable apparatus to improve its biocompatibility.

### BACKGROUND

Surgically implantable prostheses are widely used today to help patients affected by a variety of conditions including congenital, degenerative and traumatic afflictions of various body parts including, for example joints, blood vessels, heart valves and spinal tissue. The satisfactory performance of these prostheses can be affected by the level of biocompatibility with the body.

Biocompatibility is defined as the ability of a material to interface with a natural substance without provoking a biological response. In the human body, the typical response to contact with a synthetic material is the deposition of proteins and cells from body fluids on the surface of the material. The human body tolerates plastics such as PVC, polycarbonate, polyurethane and the like, for a short period of time but these materials are not considered biocompatible for long term usage.

The blood interfaces with a large surface area of the synthetic material and the initial reaction is the adsorption of a layer of protein onto these surfaces. The surface protein adsorption occurs in a short time (e.g., in minutes) and is triggered by chemical and physical phenomena related to surface features of the material and of the surrounding medium in contact with it, such as blood. The nature and quantity of this layer depends on the surface features and the composition of the blood.

Subsequently, activation of the blood and the formation of clots can set in, which may ultimately lead to thrombotic embolisation of body vessels. At present, blood coagulation is prevented or controlled by administering systemic anticoagulants, such as heparin. However, on going efforts focus on improving biocompatibility of implantable prostheses to minimize surface protein adsorption and thwart thrombus formation.

### SUMMARY

One aspect of the invention provides an implantable apparatus which has a plurality of fixed and detoxified protein layers formed in situ overlying at least one surface of the implantable apparatus.

Another aspect of the present invention provides a process of making the implantable apparatus. At least one surface of the implantable apparatus is repeatedly exposed to a protein solution to form a multi-layered coating of protein. Each of the layers of protein is fixed with a fixation solution. The multi-layered protein coating undergoes a detoxification process, such as including heparin bonding.

Yet another aspect of the present invention provides a method for improving biocompatibility of an implantable article. At least one surface of the implantable article is exposed to a protein solution so that the protein adheres to form a protein layer on the at least one surface. The protein layered implantable device is cross-linked with a fixation solution, and the implantable article is substantially detoxified and to help resist formation of thrombus on the implantable article.

Still yet another aspect of the invention provides a prosthetic device with at least one layer of a fixed and detoxified protein adhering to the surface made by a process of exposing the surface to a protein solution to form a layer of protein and fixing the protein layer by exposing the protein layer to a fixation solution and detoxifying the fixed protein layer.

### BRIEF DESCRIPTION OF THE DRAWINGS

To the accomplishment of the foregoing and related ends, certain illustrative aspects of the invention are described herein in connection with the following description and the annexed drawings. These aspects are indicative, however, of but a few of the various ways in which the principles of the invention may be employed and the present invention is intended to include all such aspects and their equivalents. Other advantages and novel features of the invention will become apparent from the following detailed description of the invention when considered in conjunction with the drawings in which:

FIGS. 1-3 illustrate a process of making an implantable apparatus according to the invention.

FIG. 4 illustrates an isometric view of an apparatus according to an embodiment of the invention.

FIG. 5 illustrates a cross-sectional view of another embodiment of the apparatus according to the invention.

FIG. 6 illustrates a cross-sectional view of yet another embodiment of the apparatus according to the invention.

FIG. 7 is a flow diagram illustrating a process of making the implantable apparatus of the present invention.

### DETAILED DESCRIPTION

Various illustrative aspects of the present invention will now be described in connection with the following figures.

The present invention provides a process to provide an implantable apparatus 10, with one or more layers 12 of a fixed and detoxified protein so as to improve its biocompatibility when implanted into the human body.

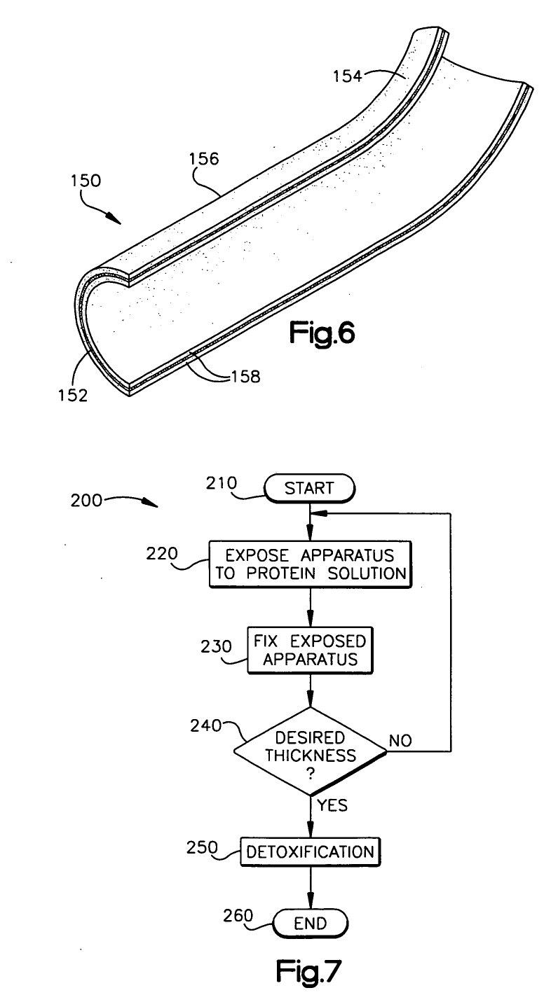

While FIG. 4 illustrates a schematic generic representation of an implantable apparatus 10 according to the present invention, such as a prosthesis, two specific exemplary embodiments are illustrated in FIG. 5 (a stent), and in FIG. 6 (a vascular graft).

Those skilled in the art will understand and appreciate that other specific examples of implantable apparatuses or prostheses may also be formed in accordance with the present invention, including, but not limited to surgical implants, and any artificial part or device which replaces or augments a part of a living body or comes into contact with bodily fluids, particularly blood. The substrates can be in any shape or form including tubular sheet, rod and articles of proper shape.

Various examples usable in accordance with the invention are known in the art. Examples include catheters, suture material, tubing, fiber membranes, bone growth stimulators, bone screws, grafts, implantable pumps, impotence and incontinence implants, intra-ocular lenses, nasal buttons, cranial implants such as cranial buttons and cranial caps, orbital implants, cardiac insulation pads, cardiac jackets, embolic devices, fracture fixation devices such as screws, nasal petal splints, nasal tampons, ophthalmic devices, periodontal fiber adhesives, staples, stomach ports, urethra stents, vaginal contraceptives, valves, vessel loops, annuloplasty rings, aortic/coronary locators, artificial pancreas, cosmetic or reconstructive surgery implants such as breast implants and facial implants, cardiac materials, such as fabric, felts, mesh, patches, cement spacers, cochlear implants, orthopedic implants, pacemakers, pacemaker leads, guide wires, patellar buttons, penile implant, prosthetic heart valves, coronary stents, vascular stents, sheeting shunts, valved conduits, joint and bone replacements such as hip bone and knee joint replacements, and spinal implants such as bone screws and inter-vertebral implants.

Turning now to FIGS. 1-3, a process for improving the biocompatibility of the implantable apparatus 10 is illustrated. The implantable apparatus 10 may be formed from a substrate 12 of a surgically useful textile material such as DACRON, polyethylene (PE) polyethylene tetraphthalate (PET), silk, Rayon, or the like or may also be formed of a surgically-useful polymeric material such as urethane, polytetrafluoroethylene (PTFE) or expanded polytetrafluoroethylene (ePTFE), or may be formed of a biological biocompatible material, such as animal pericardium (equine, bovine, porcine, etc.) or a collagen material (e.g., a remodeled collagen or fibrin biomatrix) or a surgically-useful metal such as titanium/titanium alloys, TiNi (shape memory/superelastic), aluminum oxide, platinum/platinum alloys, stainless steels. Substrates made using these materials may be coated or uncoated with a layer of a material prior to undergoing the process of FIGS. 1-3. For example, the substrate 12 can be covered with layer of a material that provides at least a portion of an outer exposed surface of the substrate, such as a polymeric material layer or a biological material layer (e.g., formed of remodeled biomatrix, such as collagen or fibrin, or animal pericardium).

The process of FIGS. 1-3 can be conducted in an ambient temperature environment, such as in a range from about 25 to about 37° C. or another other suitable temperature. The process is schematically represented in FIGS. 1-3 to include exposing the implantable apparatus 10 to a layer of a plasma protein solution 16 (FIG. 1) in a container 24 by (e.g., immersing in or flushing with) the solution for a predetermined time (e.g., 30 seconds to 2 hours). The plasma protein solution 16 may contain albumin, fibrinogen or any suitable animal plasma protein. The plasma protein solution may also contain other biomolecules such as antibacterial and antimicrobial agents; anti-inflammatories; enzymes; catalysts; hormones; growth factors; drugs; vitamins; antibodies; antigens; nucleic acids; DNA and RNA segments and proteins and peptides. The biomolecules can be synthetically derived or naturally occurring.

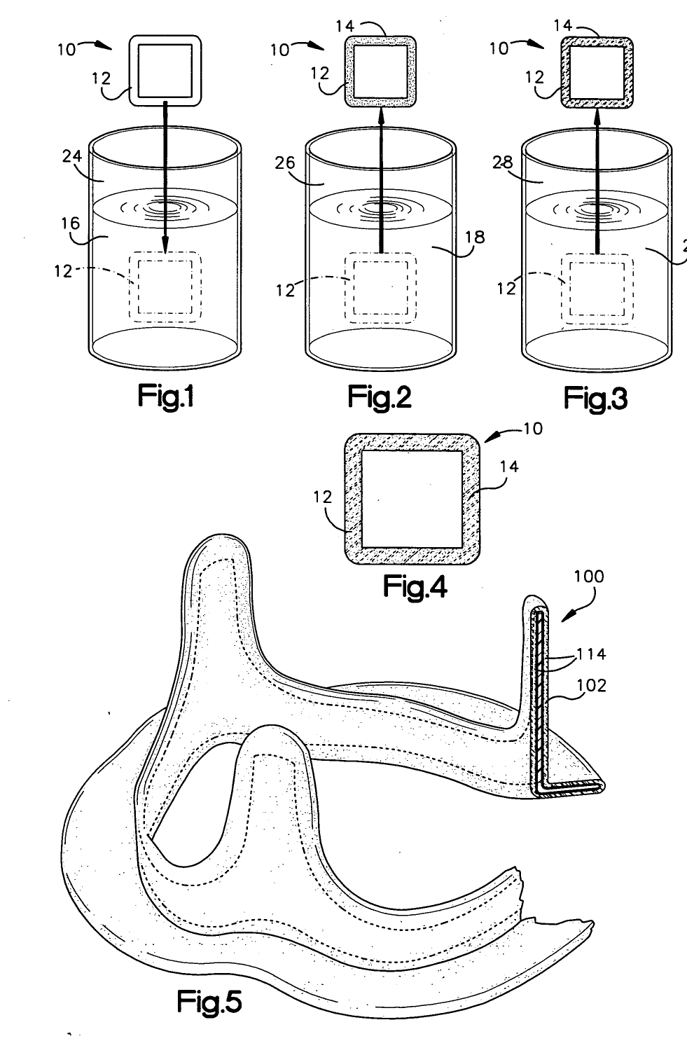

The protein coated apparatus 10 is then exposed to a fixation solution 18 in container 26 (e.g., by immersing or flushing with the solution 18) to fix the layer of plasma protein 14 relative to the substrate surface 12 of the apparatus 10 (FIG. 2). The fixation solution 18 may be an aldehyde solution, such as including glutaraldehyde and/or formaldehyde, such as at a solution concentration ranging from about 0.2% to about 1.0% aldehyde. The fixation process of FIG. 2 results in cross linking of the protein layer(s) of the apparatus 10 by binding of amine groups of the protein. Those skilled in the art will understand and appreciate suitable time periods and other fixation solutions that can be utilized according to an aspect of the present invention.

If multiple protein layers are desired, for a greater thickness, the process of soaking the apparatus 10 in the plasma protein solution 16 (FIG. 1) and fixing the plasma protein 14 to the substrate surface 12 of the apparatus 10 is repeated a desired number of times until the desired thickness is reached. Alternatively or additionally, the time period for immersion in the protein solution 16 can be increased to increase the thickness of the protein layer(s).

After a desired thickness of the protein layer(s) has been formed on the substrate surface 12, the apparatus 10 undergoes a detoxification process, which is schematically illustrated at FIG. 3. As an example, the detoxification process can include exposing the cross-linked fixed protein layer 14 to antithrombotic materials, such as a solution 20 that contains heparin or another antithrombotic solution, to inhibit the coagulation of blood by interacting with thrombin to inhibit the conversion of fibrinogen to fibrin. The detoxification renders the resulting protein layer 14 durable, non-absorbable and resistant to infection. This detoxification treatment is schematically represented in FIG. 3 by exposing the fixed protein layer 14 on the substrate 12 of the apparatus 10 to a heparin solution 20 in container 28, such as including immersing in or flushing with the heparin solution 20.

By way of example, the detoxification process can include the detoxification and heparin bonding, which is commercially available from Shelhigh, Inc., of Union, N.J., namely, the NO-REACT® process that is utilized to provide the NO-REACT® line of implantable tissue products. The detoxification further can promote covering of the substrate with endothelial cells (e.g., a thin layer of one or more cells) after the implanted apparatus has been exposed to blood.

Alternatively, prior to implanting the apparatus 10, the exposed surface (or at least the surface(s) that is to contact blood) can be seeded with a patient's own endothelial cells, as is known in the art, to promote endothelial growth. For example, endothelial cells can be extracted from a vein or other anatomic structure of the patient. The extracted endothelial cells can be cultured and grown. The cultured cells then can be seeded onto the surface of the apparatus, such as the surface portions that are to come into direct contact with blood after being implanted.

Turning now to FIG. 4, a schematic cross-sectional representation of an implantable apparatus 10 is illustrated. The implantable apparatus 10 includes at least one plasma protein layer 14 which has been fixed and substantially detoxified to improve biocompatibility, such as according to the process illustrated in FIGS. 1-3.

While the representation of FIG. 4 appears as a single layer, it will be appreciated that the protein layer 14 covering the surface of the apparatus 10 is intended to schematically illustrate any number of one or more protein layers 14 overlying the substrate surface 12 of the apparatus 10. That is, the apparatus 10 can include a plurality of protein layers or only a single protein layer to provide the apparatus 10 with a desired thickness.

The protein layer 14 can cover all of the visible surfaces of the apparatus 10. It should be understood however that under certain circumstances, it may be desirable to cover less than all of the visible surfaces of the apparatus 10 so that the surface is only partially covered with the protein layer 14.

FIG. 5 illustrates a cross-sectional view of a stent 100 according to an alternative embodiment of the invention. The particular type of stent 100 shown in FIG. 5 is for a heart valve, although the process is equally applicable to other types of stents, including, for example, coronary stents and vascular stents. The stent 100 is typically formed of a substrate of a resilient material such as plastic or metal. The stent 100 can be covered with a layer 102 of a biocompatible material, such as a textile material (e.g., a DACRON cloth) or with a biological material (e.g., remodeled collagen, remodeled fibrin, a cross-linked collagen gel, animal pericardium, dura matter, and the like).

The stent 100 includes one or more layers 114 of protein, which have been formed in-situ over an outer surface thereof. In the example of FIG. 5, the layer(s) 114 are formed overlying the surface of the exposed outer layer 102 of the stent 100. That is, the protein layer(s) 114 adhere to the layer 102 that covers the stent 100, such as by exposing the exposed surfaces of the stent to a plasma protein solution, fixing the protein layer(s) and then detoxifying the fixed protein layer(s) (e.g., according to the process of FIGS. 1-3). The entire exposed surface may be covered with covered with the protein layer 114 or alternatively, only a selected portion of the surface of the stent 100 may be covered. Additionally, those skilled in the art will understand and appreciate that the exposed surfaces of the stent can include the layer 102 as well as exposed surface portions of the underlying substrate (e.g., a metal or plastic material), such as when the layer 102 does not cover the entire stent 102.

FIG. 6 illustrates a partial cross-sectional view of a vascular graft 150 according to another alternative embodiment of the present invention. While a partial sectional view is shown in FIG. 6, those skilled in the art will appreciate that the graft 150 typically will be implemented a complete generally cylindrical member for fluidly connecting two spaces via the graft. The vascular graft 150 thus can be employed to replace a missing or degenerative blood vessel, for example. The vascular graft 150 typically includes a tubular substrate 152 of a substantially flexible material having a desired diameter and length. As an example, the material of the substrate 152 can be formed of natural or synthetic materials, such as a cloth-like material (e.g., a plastic (PTFE), DACRON or TEFLON). The substrate material can be generally porous, such that, absent processing according to an aspect of the present invention, the structure would not be sufficiently fluid-tight for use as a graft. The substrate 152 can be dimensioned and configured to provide any desired shape or length of graft 150. For instance, in the example of FIG. 6, the graft 150 includes a curved portion 154 extending arcuately from an elongated generally straight portion 156.

The vascular graft 150 includes a layer 158 of a cross-liked and detoxified protein overlying its interior and exterior surfaces 152. The protein layer 158 may include any number of one or a plurality of fixed and detoxified protein layers, such as can be formed by the process shown and described with respect to FIGS. 1-3. Alternatively, only a portion of an interior or exterior surface of the substrate 152 may be covered by the one or more layers 158.

FIG. 7 illustrates a flow diagram of a process 200 that can be employed to improve biocompatibility of an implantable apparatus. The process 200 may be automated (e.g., implemented using a conveyor belt type machine in a sterile environment). Alternatively, the process can be implemented by one or more technicians, manually and/or as part of an automated process.

The process 200 begins at 210, which can include providing one or more implantable apparatuses that are to be treated to improve biocompatibility according to an aspect of the present invention. The apparatus being treated defines a substrate for the process and includes one or more exposed surfaces. The exposed surfaces can include a surface layer of a biocompatible material, such as a fabric material (e.g., DACRON) or a biological material (e.g., a remodeled biomatrix or pericardium), such as described herein.

At 220, the apparatus is exposed to a plasma protein solution. During exposure to the plasma protein, a layer of protein adheres to the exposed one or more surfaces. The thickness of the layer formed at 220 generally will depend on, for example, the length of exposure time and the concentration of the protein in the solution. As described herein, the protein in the solution can include a combination of one or more animal proteins, such as albumin, fibrinogen or other plasma protein.

At 230, the layer of protein adhering to one or more surfaces is fixed. For example, the protein layer can be fixed by cross-linking the layer in a fixation solution, such as an aldehyde solution (e.g., glutaraldehyde or formaldehyde).

At 240, a determination is made as to whether a desired thickness of protein layer has reached a desired thickness. If the determination is made that the thickness of the protein layer has not reached a desired thickness, to process can return to 220 for repeating the exposure at 220 and fixation at 230. The process exposure and fixation can be repeated until the desired thickness is reached.

If the determination is made that the thickness has reached the desired thickness, the one or more fixed protein layers of the apparatus is substantially detoxified at 250. The detoxification can include heparin bonding of the fixed layer(s) of the apparatus. As one example, the detoxification can include heparin boding according to the NO-REACT® tissue treatment process, which is commercially available from Shelhigh, Inc., of Union, N.J. The process terminates at 260 after the apparatus has been detoxified.

From the above description of the invention, those skilled in the art will perceive improvements, changes and modifications. Such improvements, changes and modifications within the skill of the art are intended to be covered by the appended claims.

Having described the invention, the following is claimed:

1. A process to improve biocompatibility of an implantable apparatus, which comprises:
exposing at least one surface of the implantable apparatus to a protein solution to form a layer of protein on the at least one surface;
fixing the protein layered implantable apparatus with a fixation solution; and
substantially detoxifying the fixed protein layer.

2. The process according to claim 1, wherein, before the detoxification, the process further comprises repeatedly exposing the surface of the implantable apparatus to the protein solution, and fixing the protein layered implantable apparatus with the fixation solution to provide the apparatus with a multi-layered coating having a desired thickness.

3. The process according to claim 2, wherein, before the detoxification, the process further comprises:
determining whether a thickness of the fixed protein layer is a predetermined thickness;
if the thickness is not the predetermined thickness, repeatedly exposing the implantable apparatus to the protein solution to form at least one additional layer of protein; and
exposing the protein layered prosthetic device to the fixation solution.

4. The process according to claim 1, wherein the implantable apparatus further comprises a layer of at least one of a polymeric material and a biological material covering over the at least one surface of the implantable apparatus, the protein solution adhering to the layer of at least one of a polymeric material and a biological material during the exposing.

5. The process according to claim 1, wherein the protein solution comprises a solution of plasma protein.

6. The process according to claim 5, wherein the plasma protein comprises at least one of albumin and fibrinogen.

7. The process according to claim 1, wherein the fixation solution comprises at least one of formaldehyde and glutaraldehyde.

8. The process according to claim 1, wherein the implantable apparatus comprises a vascular graft.

9. The process according to claim 1 wherein the implantable apparatus comprises a stent.

10. A method for improving biocompatibility of an implantable article, the method comprising the steps of:
exposing at least one surface of the implantable article to a protein solution so that the protein adheres to form a protein layer on the at least one surface;
cross-linking the protein layered implantable article with a fixation solution; and
performing heparin bonding to detoxify the implantable article and to help resist formation of thrombus on the implantable article.

11. The method according to claim 10, wherein, before the performing, the method further comprises repeatedly exposing the surface of the implantable article to the protein solution, and fixing the protein layered implantable article with the fixation solution to provide the article with a multi-layered coating having a desired thickness.

12. The method according to claim 11, wherein, before the performing, determining whether the multi-layered coating has the desired thickness and, if the thickness thereof is not the desired thickness, repeatedly exposing the implantable article to the protein solution to form additional layers of protein, and exposing the protein layered implantable article to the fixation solution.

13. The method according to claim 10, wherein the implantable article further comprises a layer of at least one of a polymeric material and a biological material covering over the at least one surface of the implantable apparatus, the protein solution adhering to the layer of at least one of a polymeric material and a biological material during the exposing.

14. The method according to claim 10, wherein the protein solution comprises a solution of plasma protein.

15. The method according to claim 14, wherein the plasma protein comprises at least one of albumin and fibrinogen.

16. The method according to claim 10, wherein the fixation solution comprises at least one of formaldehyde and glutaraldehyde.

17. The method according to claim 10, wherein the implantable article comprises a stent.

18. The method according to claim 10, wherein the implantable article comprises a vascular graft.

19. An implantable apparatus having improved biocompatibility, the apparatus comprising:
at least one surface;
at least one layer of a fixed protein adhering to the at least one surface, the at least one layer of fixed protein having been formed by exposing the surface to a protein solution to form the at least one layer of protein and by fixing the at least one layer of the protein and by substantially detoxifying the fixed protein layer, which detoxification includes heparin bonding.

20. The implantable apparatus according to claim 19, further comprising a plurality of layers of the fixed protein formed over the at least one surface by repeatedly (i) exposing the at least one surface to a protein solution and (ii) fixing the protein layers with a fixation solution.

21. The implantable apparatus according to claim 19, wherein the protein solution comprises a plasma protein.

22. The implantable apparatus according to claim 21, wherein the plasma protein comprises at least one of albumin and fibrinogen.

23. The implantable prosthetic device according to claim 19 wherein the fixing of the protein layer further comprises fixing the at least one layer of the protein in fixation solution that comprises at least one of formaldehyde and glutaraldehyde.

24. The implantable prosthetic device according to claim 19, further comprising a layer of at least one of a polymeric material and a biological material that covers at least a substantial portion of an underlying substrate of the implantable apparatus to provide the at least one surface thereof, the at least one layer of the fixed protein adhering to the at least one surface, which includes the layer of at least one of a polymeric material and a biological material.

25. An implantable apparatus having improved biocompatibility comprising:
at least one surface; and
a plurality of protein layers formed in situ overlying the at least one surface, the plurality of layers having been fixed and substantially detoxified.

26. The implantable apparatus according to claim 25, wherein the detoxified plurality of layers in a detoxification process that includes heparin bonding.

27. The implantable apparatus according to claim 25, wherein the at least one surface further comprises a layer of polymeric material interposed between the at least one surface and the plurality of layers, the plurality of layers adhering to the layer of the polymeric material.

28. The implantable apparatus according to claim 25, wherein the plurality of protein layers comprises at least one of albumin and fibrinogen.

29. The implantable prosthetic device according to claim 25, further comprising an layer of at least one of a polymeric material and a biological material that covers at least a substantial portion of an underlying substrate of the implantable apparatus to provide the at least one surface thereof, the plurality of protein layers adhering to the at least one surface, which includes the layer of at least one of a polymeric material and a biological material.
